(19) United States
(12) Patent Application Publication
(10) Pub. No.: US 2009/0137477 A1
(43) Pub. Date: May 28, 2009

(54) Treatment of oropharyngeal candidiasis in cancer patients

(75) Inventor: Pierre Attali, Vincennes (FR)

Correspondence Address: BIRCH STEWART KOLASCH & BIRCH, PO BOX 747, FALLS CHURCH, VA, 22040-0747, US

(73) Assignee: BIOALLIANCE PHARMA, Paris (FR)

(21) Appl. No.: 12/289,925

(22) Filed: Nov. 6, 2008

Related U.S. Application Data
(60) Provisional application No. 60/986,688, filed on Nov. 9, 2007.

(51) Int. Cl. A61K 38/16; A61P 43/00

(52) U.S. Cl. 514/12

(57) ABSTRACT

A method for local treatment of severe oral lesions caused by oropharyngeal candidiasis in immunocompromised patients, in particularly having head and neck cancer and undergoing or have undergone radiation therapy, by administering a prolonged release mucoadhesive tablet containing at least one active principle of miconazole. Methods of treating xerostomia in cancer patients having severe oral lesions is also provided.

This Nonprovisional application claims priority under 35 U.S.C. § 119(e) on U.S. Provisional Application No(s). 60/986,688 filed on Nov. 9, 2007, the entire contents of which are hereby incorporated by reference.

### FIELD OF THE INVENTION

The present invention relates to a method of treating oropharyngeal candidiasis in cancer patients having severe oral lesions using a prolonged release mucoadhesive tablet. More specifically, the present invention relates to a method of treating severe oral lesions associated with a Candidiasis in the mouth of head and neck cancer patients undergoing radiation therapy using a prolonged release mucoadhesive tablet containing miconazole as the active principle. The present invention also relates to treating xerostomia in cancer patients having severe oral lesions using the same prolonged release mucoadhesive tablet.

### BACKGROUND OF THE INVENTION AND RELATED PRIOR ART

Candidiasis is a fungal condition that is caused by yeast called Candida albicans and is commonly known as thrush. Besides Candida albicans, Candida krusei, Torulopsis glabrata or Candida tropicalis are sometimes implicated in the cause of thrush in AIDS and cancer patients.

Candida is present in the oral cavity of 40% to 60% of healthy humans and is not harmful, since in healthy people their immune system can keep this fungal condition suppressed. However, in immunocompromised patients, it can become aggressive, induce oropharyngeal candidiasis (OPC), invade the digestive tract and even lead to systemic infections (1).

There are three different types of oral candidiasis, which have different clinical presentations. Pseudomembranous candidiasis in which adherent white plaques appear on the buccal mucosa and the mucosa of the throat, tongue or gingivae. These white discrete plaques can be wiped off. Erythematous candidiasis in which red macular lesions appear as red patches on the hard or soft palate, the dorsum of the tongue or in the buccal mucosa. Hyperplasti candidiasis which results in white adherent patches or plaques usually found bilaterally on the buccal mucosa.

Oropharyngeal candidiasis (OPC) is the most common oral fungal infection in cancer patients (2-5), with a mean incidence between 30 to 70% (6). Its occurrence is favoured by chemo and/or radiation therapy-induced xerostomia and mucositis (7). Decreased salivary secretion can lead to xerostomia, which can interfere with speech and swallowing, can cause fetid breath and due to the reduced salivary flow, bacteria can no longer be washed away thus impairing oral hygiene. In cancer patients, it results in oral discomfort and marked reduction in food and liquid consumption, contributes to poor nutritional status and strongly deteriorates the quality of life (6).

Patients with head and neck cancer are particularly susceptible to oropharyngeal candidiasis since they usually suffer from both markedly reduced salivary secretion and radiation therapy-induced mucositis (8-13). The occurrence of oropharyngeal candidiasis (OPC) in these patients further deteriorates oral mucosa, increases pain, dysphagia, and anorexia, alters taste and contributes to a worsening nutritional and general health state. Cancer patients can experience mild to excruciating pain due to oropharyngeal candidiasis for a variety of reasons. Recurrence occurs in approximately one-third of patients.

Radiation therapy for head and neck cancer substantially affects the condition of oral cavity and includes mucositis, deteriorated functioning of salivary glands and risk of fungal infection. These may potentially be causing pain upon eating and possibly leading to temporary suspension of radiation therapy, for up to 15 days (14). These effects are aggravated if radiation therapy is combined with chemotherapy (14). Oropharyngeal candidiasis is the most frequent infection in those patients (13).

The incidence of Candida colonisation reaches 93% and that of oral candidiasis varies widely from 17% to over 29% Candida colonisation in head and neck cancer patients peaks 6 months after radiation therapy and slowly declines to above normal values after 18 months (15). Patients, especially when they suffer from associated radiation-induced mucositis, remain at risk of oropharyngeal candidiasis during this period. The occurrence of OPC increases the risk of treatment-induced complications, delays cancer treatment and further worsens the local and general patient state. It therefore needs to be treated.

The objectives of the treatment of oropharyngeal candidiasis (OPC) are to eliminate signs and symptoms of the disease and to prevent recurrence. The Infectious Disease Society of America recommends to treat initial episodes of OPC with topical treatments and to reserve systemic treatments to frequently recurrent episodes of oropharyngeal candidiasis (OPC) and to oesophageal dissemination (16). In practice, these recommendations are not followed mainly because of the need for 4 to 6 daily doses and the poor taste acceptance of topical agents that compromise patient compliance and may lead to poor to moderate efficacy. Consequently, despite their risk for drug-drug interactions and liver toxicity, once daily dosing azole agents that improve patient compliance have progressively replaced topical drugs.

The large use of fluconazole has recently been incriminated in the selection and development of resistant strains of Candida and in the increase in the incidence of non-albicans species (17, 18). There is therefore a need for a topical agent that is as effective as systemic agents particularly for the treatment of severe Candidiasis, well tolerated, administered once daily, and does not increase the risk of emergence of resistance.

Intraconazole oral solutions and clotrimazole troches have also been used to treat oropharyngeal candidiasis in immunocompromised patients. However, gastrointestinal side effects such as nausea, diarrhea and abdominal pain were experienced in one fourth of the patients treated (19).

Miconazole possesses a broad spectrum antifungal activity against the most frequent Candida observed in oropharyngeal candidiasis (OPC) including C. glabrata, C. krusei and C. tropicalis (20, 21). Interestingly, no initial resistance has been described and resistance in chronically treated patients exceptionally reported. Topical miconazole is hardly used in OPC because of multiple daily dosing and bad taste (22).

Thus, other solutions were sought to treat antifungal infections caused by Candida. For example, U.S. Pat. No. 6,673,373 describes antifungal formulations using azole antimicrobial drugs such as intraconazole, saperconazole, ketoconazole and fluconazole in the form of a pharmaceutical composition. The pharmaceutical compositions are in the form of pellets which comprise a core and a drug emulsion layer and optionally a protective layer. The pharmaceutical provides superior solubility and availability.

A method of treating an azole-resistant fungal infection caused by azole-resistant strains by using a composition containing a squalene epoxidase inhibitor terbinafine and an azole 14-alpha-methyldemethylase inhibitor such as fluconazole or itraconazole is described in U.S. Pat. No. 5,985,906.

Although various antifungal treatments were known in the art using azole antifungal drugs only topical treatments for cancer patients being treated with radiation therapy were recommended for oropharyngeal candidiasis due to the problem of toxicity and resistance. But these topical treatment are not completely efficient for treatment of severe Candidiasis.

Thus, it is an object of the present invention to treat severe Candidiasis with a topical treatment and therefore overcome the problems associated with treating oropharyngeal candidiasis in immunocompromized patients.

It is another object of the present invention to provide a method of treating cancer patients having severe oral lesions associated with oropharyngeal candidiasis. undergoing or having undergone radiation therapy.

It is another object of the present invention to provide a method of treating cancer patients having severe oral lesions associated with oropharyngeal candidiasis using a prolonged release mucoadhesive tablet with as little side effects as possible.

It is another object of the present invention to provide a method of treating severe oral lesions associated with a Candidiasis in the mouth of patients with head and neck cancers which have undergone or are undergoing radiation therapy using a prolonged release mucoadhesive tablet containing at least one active principle wherein said at least one active principle is miconazole.

Yet another object of the present invention relates to treating xerostomia in cancer patients having severe oral lesions using the above described prolonged release mucoadhesive tablet.

In yet another object the present invention provides preventing the reoccurrence of Candidiasis in patients with head and neck cancer and having undergone or undergoing radiation therapy and having severe oral lesions associated with oropharyngeal candidiasis.

These and other objects are achieved by the present invention as evidenced by the summary of the invention, description of the preferred embodiments and the claims.

### SUMMARY OF THE INVENTION

The present invention thus provides a method for local treatment of oropharyngeal candidiasis in immunocompromised patients having severe oral lesions said method comprising administering to a patient in need of such treatment a buccal mucoadhesive tablet having at least a sustained release and comprising at least one principle active, quantities of natural proteins representing at least 50% by weight of active principle wherein said at least one active principle is miconazole.

The present invention furthermore provides a method for treating oropharyngeal candidiasis in cancer patients having severe oral lesions said method comprising administering to those cancer patients in need of such treatment a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein at least one active principle is miconazole.

In another aspect the present invention a method for treating oropharyngeal candidiasis in cancer patients having severe oral lesions, wherein the at least one active principle is miconazole present in the amount of between 30% to 50% (w %/w %), the natural proteins are milk proteins present in an amount of from 18% to 28% (w %/w %), the alkali metal alkylsulphate is sodium lauryl sulphate present in an amount of 3.5% to 5.50% (w %/w %), the compression excipient is corn starch, the hydrophilic polymer is hydroxypropylmethycellulose and the monohydrate sugar is lactose monohydrate.

In yet another aspect a method for preventing the relapse of severe oral lesions caused by oropharyngeal candidiasis in a cancer patient having undergone or undergoing radiation therapy, the method comprising administering to those cancer patients in need of such treatment: a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole is provided.

Yet another aspect of the present invention provides a method of treating xerostomia in cancer patients having severe oral lesions, said method comprising: administering to those cancer patients in need of such treatment a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole.

In another aspect the methods described above and herein for treating oropharyngeal candidiasis in cancer patients having severe oral lesions are for treating head and neck cancer patients that have undergone or are undergoing radiation therapy.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1 is a flow chart illustrating the preparation of a miconazole tablet according to the present invention.

FIG. 2 is a diagram of the disposition of patients in the modified intent-to treat (mITT) population and in the safety population.

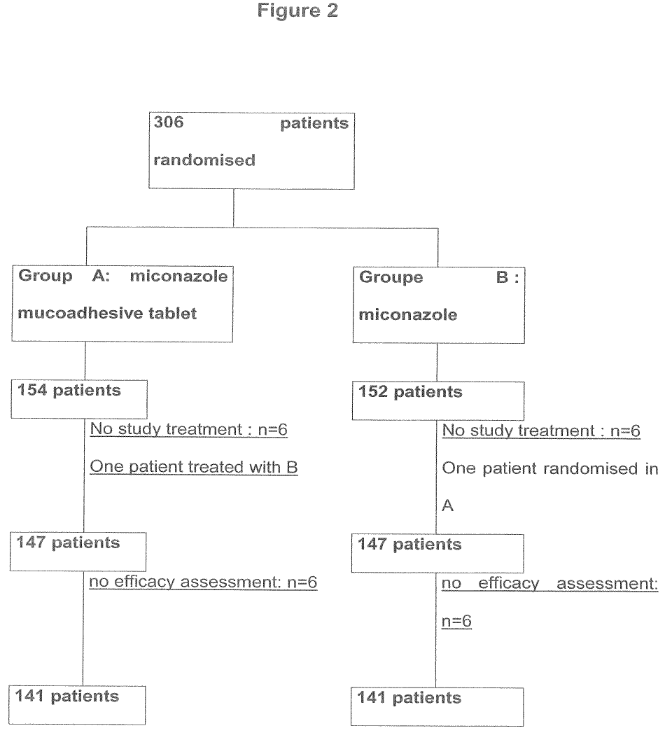

FIG. 3 is a picture of severe oral lesions caused by oropharyngeal candidiasis.

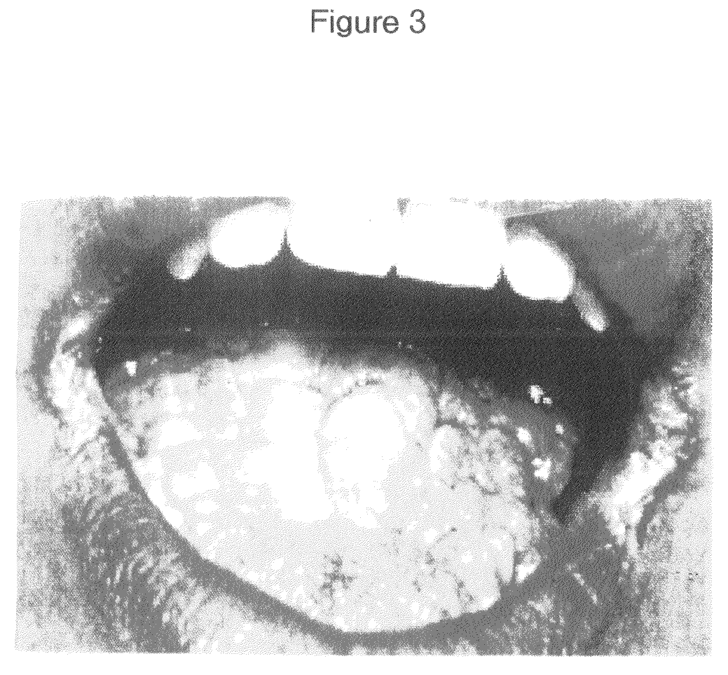

### PREFERRED EMBODIMENTS OF THE INVENTION

As used herein “severe oral lesions” means lesions associated with or caused by erythematous/atropic acute candidiasis or chronic hyperplastic candidiasis. The lesions caused by erythematous/atropic acute candidiasis present as red lesions and can be unique lesions, multiple lesions, which are localized or spread, confluent red lesions and/or combinations thereof. Hyperplastic chronic candidiasis lesions present as white persistent lesions which cannot rub off.

Severe oral lesions are those that are described in Muarray et al (19).

The term “oral” includes the tongue cheeks and palate, as well as the pharynx, which is that section of the alimentary canal that extends from the mouth and nasal cavities to the larynx, where it becomes continuous with the esophagus.

The term “treat” encompasses reducing totally or at a high percentage the severe oral lesions. Thus, complete or partial disappearance of severe oral lesions in the oral cavity after treatment with the prolonged release mucoadhesive tablet of the present invention is achieved or the lesions are treated to the extent that there are some oral lesions remaining but the severe symptoms encountered by the patient are eleveated.

The term “immunocompromised” means having an immune system that has been impaired by disease, in particular by cancer and HIV, or treatment.

The term “relapse” means that the patient does not have oropharyngeal candidiasis after previous treatment after a period of at least 4 weeks.

By “prolonged release” is meant that the active principle in the mucoadhesive tablet is released immediately after 30 minutes and then over a prolonged period of time of at least 6 hours, or at least 12 hours or at least 24 hours.

“Head and neck cancers” encompass cancers of the mouth, nose sinuses, salivary glands, throat, larynx and lymph nodes in the neck.

As used herein “radiation therapy” means the use of any ionizing radiation to kill cancer cells and to shrink tumors.

As used herein, “xerostomia” encompasses conditions of not having enough saliva in the mouth.

The present invention thus provides a method for local treatment of oropharyngeal candidiasis in immunocompromised patients having severe oral lesions said method comprising administering to a patient in need of such treatment a buccal mucoadhesive tablet having at least a sustained release and comprising at least one principle active, quantities of natural proteins representing at least 50% by weight of active principle wherein said at least one active principle is miconazole.

The present invention thus provides a method for treating oropharyngeal candidiasis in cancer patients having severe oral lesions said method comprising administering to those cancer patients in need of such treatment a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein at least one active principle is miconazole.

Besides a method for treating oropharyngeal candidiasis in cancer patients having severe oral lesions, the present invention also provides a method for treating the relapse of severe oral lesions caused by oropharyngeal candidiasis in a cancer patient having undergone or undergoing radiation therapy, the method comprising administering to those cancer patients in need of such treatment: a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole.

In another aspect, the present invention relates to a method of treating xerostomia in cancer patients having severe oral lesions, said method comprising, administering to those cancer patients in need of such treatment: a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole.

The prolonged release mucoadhesive tablets of the present invention contain natural proteins that confer adhesiveness and which represent at least 50% by weight of the active principle. These natural proteins are those described in EP 0 542 824. Examples of such natural proteins include milk protein concentrates titrating at a minimum of 85% proteins such as Prosobel L85, LR85F or Promilk 852A sold by Armor Proteins. Other natural proteins that can be used in the mucoadhesive tablet are those from the Alaplex range of 4850, 1180, 1380 or 1395 from NZMP.

Natural proteins of vegetal origins can also be used in the present invention such as natural pea proteins, natural soy proteins, natural potato proteins, natural wheat proteins, gliadin proteins and mixtures thereof. The method for producing pea protein is described in WO 2007/017571. Examples of pea proteins are those titrating a minimum of 85% proteins such as Nutralys® sold by Roquette (France). Pea protein is particularly rich in lysine and has high levels of branched amino acids, glutamic acid and arginine.

The relative concentration of natural proteins in the mucoadhesive tablet is between 15% to 50% (w %/w %). In another aspect the natural proteins are in a concentration of 20% to 30% (w %/w %).

The hydrophilic proteins can be selected among those well known in the art such as hydroxypropylmethylcellulose, polyacrylic acid, carbomer, hydroxyethylcellulose, HPMC, carboxymethylcellulose, PVA, cellulose ethers, xanthum gum, scleroglucan, locust bean gum, gum Arabic, gum tragacanth, carob, alginic acid, alginates, carrageenates, agar-agar and guar gum either alone or in mixtures thereof. Other polymers that can be used in the present invention include cellulose based polymers such as hydromellose, cellulose acetate, cellulose esters, cellobiose, cellulose resins alone or in mixtures thereof.

Compression agents are added to reinforce the hardness of the prolonged release mucoadhesive tablet. The compression agents that can be used in the formulation of the tablet include flow aids including talc, colloidal silicone dioxide, colloidal silica and lubricants including magnesium stearate, stearic acid, polyethylene glycol can also be added to the bioadhesive slow release carrier at the stage of blending, as well as corn starch.

These additional agents can be added to the carrier in the concentration range of 0.1 to 10% by weight of the total weight of the components in the prolonged release mucoadhesive tablet.

An alkali metal alkylsulfate is also a component of the bioadhesive carrier of the present invention. This alkali metal sulfate increases the granulation of the active principle acting as a solubilization agent. The alkali metal alkylsulfate that can be used in the formulation includes magnesium lauryl sulfate, potassium lauryl sulfate, sodium laurylsulfate and diethylsulphosuccinate. Generally it is present in the prolonged release mucoadhesive tablet at a concentration of between 1 to 10% by weight or 2 to 10% by weight.

The adhesive system can also comprise a diluent. This diluent is a monohydrate sugar. The diluent is present in an amount between 0.1% and 1% by weight in the mucoadhesive tablet. The monohydrate sugar can be either lactose monohydrate or saccharose. In one aspect of the present invention the monohydrate sugar is lactose monohydrate.

The mucoadhesive tablet used in the present invention permits liberation of the active principle, preferably a prolonged and/or immediate and/or local liberation of the active principle. More specifically this tablet is described in U.S. Pat. No. 6,916,485, which is incorporated herein by reference.

In one aspect of the present invention the at least one active principle is miconazole, which is (RS)-1-[-2-(2,4-dichlorobenzyloxy)-2(2,4-dichlorophenyl)-ethyl]-1H-imidazole and is present in a dose of 10 mg to 150 mg per tablet. In another aspect, miconazole, as an active principle is present in a dose of 25 to 75 mg. In yet another aspect the miconazole is present in an amount of 50 mg per prolonged release mucoadhesive tablet.

In addition to miconazole, the prolonged release mucoadhesive tablet of the present invention can be formulated with a member of the broad spectrum azole family including clotrimazole, ketoconazole, fluconazole, itraconazole, isoconazole, econazole, saperconazole, genaconazole, terconazole, butoconazole, tioconazole, oxiconazole, bifonazole, fenticonazole, omoconazole, sertaconazole, voriconazole, and sulconazole.

The azole miconazole type compound can be associated with yet a further active principle such as an antifungal with a different spectrum such as a polyene, an analgesic, a salivation agent, a saliva substitute, an antiseptic, anaesthetic compounds, an anti-inflammatory, thalidomide or mixtures thereof.

Examples of broad spectrum polyenes that can be used in the present invention include tetraenes, nystatin and amphotericin B. The doses of polyenes that can be used in the mucoadhesive tablet range from 10 to 100 mg. For example, amphotericin can be used in the dosage ranging from 20 to 60 mg, while nystatin can be used in an amount of 10 mg to 100 mg.

Analgesics that can be included in the prolonged release mucoadhesive tablet are aspirin, paracetamol, ibuprofen and naproxen. Depending upon the pain encountered by the cancer patients, opioids such as codeine, Darvocet, Demerol and Percocet can also be added to the prolonged release mucoadhesive tablet of the present invention.

The dosage of the analgesic may vary depending on the patient and the amount of pain that the patient is suffering from.

Examples of salivation agents that can be used in the mucoadhesive tablet include pilocarpine and bethanechcol. The dose of the salivation agent is generally 0.1% to 10% by weight of the totals weight of the active constituents in the mucoadhesive tablets of the invention. Lower dosages of between 0.1% and 5% are also contemplated for use in the prolonged release mucoadhesive tablet of the present invention.

Sodium laurylsulfate, iodophore, iodine, chlorhexidine gluconate, quaternary ammonium compounds such as cetylpyridinium chloride, phenolic antiseptics such as Listerine®, povidone-iodine, hexetidine, triclosan, delmopinol, salifluor, sanguinarine, alkali metal alkylsulfates and propolis are antiseptics that can be used in the present invention.

Between 0 to 5% of the antiseptic is used in the bioadhesive carrier of the invention.

Anaesthetics can be used since severe oral lesions called by oral oropharyngeal candidiasis is often accompanied by severe oral pain. Examples of anaesthetics that can be used are lidocaine or tetracaine and can be used in the range of 0.1% to 10% of the total weight of the active ingredients of the composition.

Examples of anti-inflammatory drugs that can be used in the bioadhesive carrier include aspirin, salsalate, diflunisal, ibuprofen, ketoprofen, nabumetone, piroxicam, naproxen, diclofenac, indomethacin, sulindac, tolmetin, etodolac, ketorolac, oxaprozin, trisalicylate, acetaminophen, suprofen, corticoids, celecoxib and thalidomide.

The dose of the anti-inflammatory present in the bioadhesive slow release carrier is between 25 and 1,500 mg or between 50 and 500 mg depending on the anti-inflammatory utilized.

Binders can also be added in the formulation of the prolonged release mucoadhesive tablet. The binders used in the present invention can be selected from carboxy vinyl polymer, methycellulose, hydroxyethylcellulose, hydroxypropyl cellulose, polyvinylpyrrolidone, polyethylene glycol and the like. The binders are present in the amount of 0.5 to 5% by weight in the bioadhesive slow release carrier.

Also flavour masking agents can be used, if necessary, to mask the disagreeable flavour of the prolonged release mucoadhesive tablet. Due to the low dose of miconazole in the mucoadhesive tablet of around 50 mg a flavour masking agent is not really necessary since at this low dosage, there is an absence of disagreeable flavour. However, if the dosage is increased or if another antifungal is used that has a disagreeable flavour a flavour masking agent such as a menthol or menthol derivative can be formulated in the prolonged release mucoadhesice tablet.

The carrier of the present invention can be in the form of a tablet, microspheres and the like. They can be formulated in any shape such as rectangular, circular, square, oval and the like. For example, for gingival delivery, the carrier has a flat surface and a curved surface. The carrier can also be coated with the active principle.

The prolonged release mucoadhesive tablets are generally administered once a day. In another aspect the tablets can be administered bucally two times a day. The period of administration can be from at least 7 days to 14 days. This period can be longer if the severe oral lesion is not fully treated after 14 days.

In one aspect, the prolonged release mucoadhesive tablet contains 50 mg of miconazole and is administered bucally once daily for fourteen days. In severe cases of oral lesions the mucoadhesive tablet can be administered beyond 14 days and, for example, between 14 days and 3 months depending upon the severity of the oral lesions.

Although contemplated in a 50 mg dose at least miconazole as the active principle or miconazole and at least one composition from the broad spectrum azole family, the active principle of the present invention can be administered in doses that are less than 50 mg or more than 50 mg depending on the condition of the oral lesions in the mouth of the cancer patient. For instance, when the severe oral lesions caused from oropharyngeal candidiasis result in symptoms in a patient that can effect the radiation therapy scheduled to aid in the treatment, diminution or treatment of the cancer, the dose may be increased to over 50 mg per day. In one aspect under these conditions the dose may be between 55 mg to 150 mg per day. In another aspect the prolonged release mucoadhesive tablets can be in a dose of between 60 mg to 450 mg per day.

Likewise the dosage may be decreased to less than 50 mg depending on the condition of the cancer patient.

It should be appreciated that by eliminating the high quantity of miconazole that is delivered by the prolonged released mucoadhesive tablet of the present invention undesirable side effects are diminished since less amounts are used avoiding the symptoms associated with high concentrations such as nausea, vomiting and the like. Thus it would be advantageous to deliver a dosage of the miconazole and at least one active ingredient from the broad spectrum azole family at a dosage of around 50 mg.

Furthermore the prolonged release mucoadhesive tablets of the present invention are advantageous in that the minimum inhibitory concentration (MIC) is achieved over a longer period of time.

Moreover, the salivary concentration of the prolonged release mucoadhesive tablets of the present invention are superior to 1 μg/ml for at least 7 hours.

The severe oral lesions predominant in cancer patients undergoing radiation therapy that can be treated by the method of the present invention are caused from unique lesions, multiple lesions, which are localized or spread and confluent red lesions. These lesions thus can be a sole lesion, being either red or white that cannot be rubbed off, which is in the oral cavity, multiple lesions of least 2 and between 2 to 8 lesions in the oral cavity and have the same coloring as those described above for a single oral lesion and/or spread and confluent oral lesions, which are those which lesion cover in different places throughout the oral cavity and are connected in “stream-like” patterns throughout the oral cavity. Severe lesions are those defined in Murray et al (19).

Moreover, there is 55% to 90% cure rate of the severe oral lesions and clinical symptoms improved in 75% to 90% of the cases.

In yet another aspect the present invention also relates to the use of the above described prolonged release mucoadhesive tablets described in detail above for the production of a medicament to treat severe oral lesions caused by oropharyngeal candidiasis in cancer patients undergoing or undergone radiotherapy.

In yet another aspect the present invention relates to the use of the above-described prolonged release mucoadhesive tablet described in detail above for the production of a medicament to prevent relapse or xerostomia of severe oral lesions caused by oropharyngeal candidiasis in cancer patients undergoing or undergone radiotherapy.

A number of embodiments of the invention have been described. Nevertheless, it will be understood that various modifications may be made without departing from the spirit and scope of the invention.

### EXAMPLES

### Example 1

### Preparation of a Tablet Containing 50 mg of Miconazole

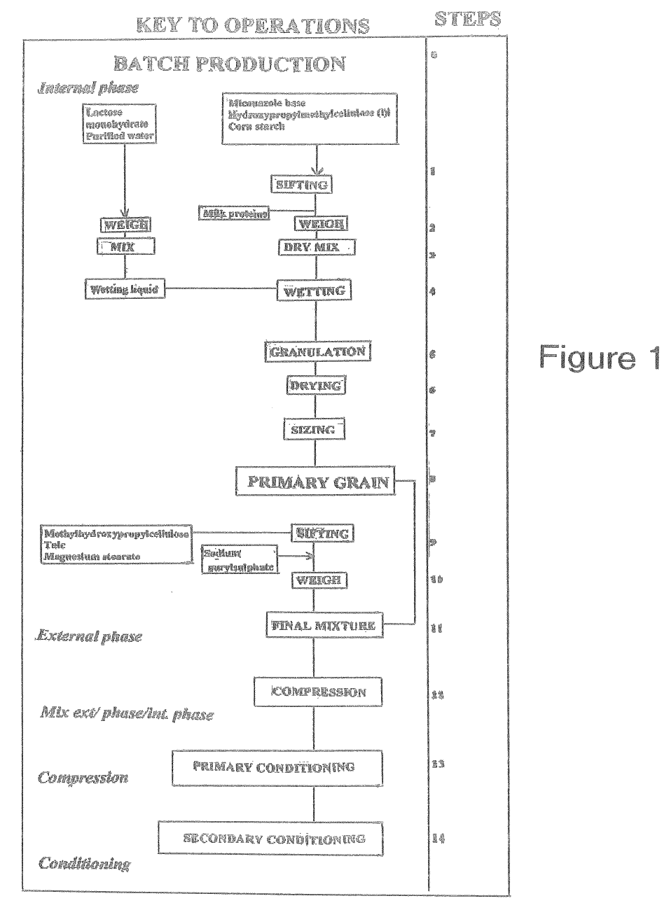

A flow chart for the preparation of the miconazole tablet is shown in FIG. 1. 50 mg of miconazole base (or 100 mg for a 100 mg tablet) was mixed with 9.2 mg of corn starch and 20.5 mg of methylhydroxypropylcellulose.

The mixture was then homogenized by sieving and 27.43 mg of LR85F milk proteins was added and mixed with the initial mixture. This latter was then wetted with a mixture of lactose monohydrate (lactose 200 Mesh in an amount of 0.39 mg in purified water). During batch production, all of the wetting liquid was introduced, namely 0.39 mg per 115 mg tablet, representing 0.34% by weight of total constituents. The wetting step was followed by a granulation, drying and sizing step carried out under conventional conditions, to obtain the primary grain.

A mixture of excipients was added to this primary grain: talc, magnesium stearate, methylhydroxypropylcellulose, to which sodium laurylsulphate in a concentration of 4.5% was added after sieving.

The final mixture then underwent conventional phases of tablet compression and packaging.

The composition of a 50 mg dose tablet is shown in Table 1 below.

TABLE I

| | 50 mg dose batch | |
|---|---|---|
| | composition in mg/tablet | Percentage |
| miconazole base | 50.00 | 43.50 |
| methylhydroxypropylcellulose | 20.50 | 17.80 |
| Metolose 90SII150000 | | |
| LR85F milk proteins | 27.43 | 23.80 |
| corn starch | 9.20 | 8.00 |
| lactose monohydrate (lactose | 0.39 | 0.34 |
| 200 mesh) | | |
| sodium laurylsulphate | 5.18 | 4.50 |
| magnesium stearate | 1.32 | 1.15 |
| Talc | 0.98 | 0.85 |
| Purified water | qs | |
| TOTAL | 115.00 | 100 |

In this composition, the milk proteins could be selected from all of the concentrates cited above provided that they are titrated to at least 85% proteins.

Further, 29.59 g±3 per tablet of saccharose could be substituted for 0.39 g per tablet lactose.

### Example 2

### Scale-Up 10 kg Batch

Industrial scale-up was carried out with the same composition as set forth above in Table I to produce a 10 kg batch.

On a large scale, the method of FIG. 1 can be adapted and simplified by introducing a fixed quantity of lactose, without compensating with metolose; and by carrying out a previous calculation of the quantity of water to be introduced, which meant that the grain appearance could be ignored, rendering the method adaptable to a closed vessel apparatus.

### Example 3

### Patient Selection

Male and female patients aged >18 years having undergone radiation therapy for head and neck cancer with clinical signs and symptoms of oropharyngeal candidiasis who had either a first episode or a relapse of oral candidiasis were eligible for the study. Oropharyngeal candidiasis was confirmed by direct mycological examination and positive fungal culture with >100 colonies.

More specifically the direct mycological examination involved taking samples form the mouth by scraping, culturing them and microscopically examining the cultures for the presence of Candida albicans and Candida non-albicans.

Patients were excluded if they had systemic or disseminated candidiasis outside the oropharyngeal cavity, had received systemic antifungal treatment within the last 14 days, were receiving concomitant treatments that may interfere with miconazole (anticoagulants, hypoglycaemic sulfonylureas), had liver failure defined as AST and/or ALT >5 times the upper limit of the normal values (ULN) and/or reduced prothrombin time (<80%) or were unable to understand or follow the study procedures. Exclusion criteria also included poor general condition (ECOG >2), recent participation in another trial, and females of childbearing age with no acceptable contraception methods or treatment.

The study was approved by independent ethics committees and was performed in accordance with Good Clinical Practice, guidelines of ICH and the Declaration of Helsinki. All patients signed a written informed consent before participation in the study.

Eligible patients were randomized to receive either miconazole Lauriad 50 mg muco-bioadhesive tablet (MBT—Loramyc®, BioAlliance Pharma) once daily or MOG 500 mg (Daktarin®, Janssen-Cilag) daily in four 125 mg divided doses for 14 days. Patients were instructed to apply the MBT on the gum, once daily in the morning. MOG was to be kept in the mouth as long as possible before swallowing.

A total of 306 patients were enrolled in the study from May 2002 to June 2004; 282 patients (141 per treatment group) were included in the mITT population and 294 patients (147 per treatment group) in the safety population (Error! Reference source not found.2). The baseline characteristics for the mITT population are presented Table II. Patient demographics, characteristics and disease history were not different between the two treatment groups. However, the percentage of patients with reduced or absent salivary secretion and with multiple or confluent oral lesions were marginally higher in the MBT (21% and 71% respectively) than those observed in the gel group (13% and 58%, p=0.09 and 0.06 respectively). Pre-treatment samples determined that C. albicans was the only causative organism in 63% of patients while C non-albicans were the only species in 25% of patients.

Table II below describes the baseline characteristics of 282 modified intent-to-treat patients who have undergone radiation therapy for head and neck cancer and who have oral candidiasis.

TABLE II

| | Miconazole 50 mg | |
|---|---|---|
| | Mucoadhesive Buccal | Miconazole 500 mg |
| | Tablet | Oral Gel |
| | (n = 141) | (n = 141) |
| Men | 73% | 77% |
| Women | 27% | 23% |
| Age (y), Median (range) | 53 (17-83) | 54 (17-83) |
| ECOG 0 | 63.1% | 67.4% |
| ECOG 1 | 33.3% | 27.6% |
| ECOG 2 | 3.5% | 5% |

Localization

| | | |
|---|---|---|
| Cavum | 46.1% | 44% |
| Oropharynx | 7.8% | 9.2% |
| Larynx | 6.4% | 9.2% |
| Not specified | 39.7% | 37.6% |

Treatment

| | | |
|---|---|---|
| Surgery | 31.9% | 37.6% |
| Chemotherapy | 66% | 67.4% |
| Radiotherapy | 100% | 100% |
| Mean delay (months) | 13.5 | 11.5 |
| Range (months) | TBC | TBC |

Oropharyngeal candidiasis characteristics

| | | |
|---|---|---|
| History of antifungal | 50.3% | 49.6% |
| treatment | | |
| Local | 33.3% | 34.7% |
| Systemic | 17.0% | 14.9% |
| Salivary secretion* | | |
| 1. - Absent | 21.3% | 13.5% |
| 2. - Partial | 74.5% | 82.3% |
| 3. - Normal | 4.3% | 4.3% |
| Extent of lesion** | | |
| 1 - Unique localized | 34.8% | 40.4% |
| 2 - Multiple localised | 53.2% | 45.4% |
| 3 - Spread or confluent | 12.1% | 14.2% |
| Burning/soreness | 50.4% | 51.1% |
| Odynophagia | 36.9% | 39% |
| Inflammation | 72.3% | 73% |
| Erythema | 73.8% | 78.8% |
| Mucositis | 53.9% | 56% |
| Mild | 39.7% | 38.3% |
| Moderate | 13.4% | 15.6% |
| Severe | 0.7% | 2.1% |

Concomitant treatments and disease

| | | |
|---|---|---|
| Diabetes | 0% | 2.8% |
| Corticosteroids | 2.8% | 4.2% |
| Antibiotics | 5.6% | 7% |

ECOG = Eastern Cooperative Oncology Group
*p = 0.09
**p = 0.06 between groups

### Example 4

### Study Procedures & Endpoints

Baseline assessments were carried out on day 1 (see, Table II above), efficacy assessments on days 7 and 14, and recurrence assessments on days 30 and 60. At all visits, the Murray Scoring Scale was used to evaluate the extent of the oral candidiasis lesions (0=none; 1=single localised; 2=multiple localised; 3=extensive or confluent). Candidiasis symptoms (odynophagia, burning/soreness) were evaluated using a 0-3 rating scale where 0 was absence of symptoms, 1 mild, 2 moderate, and 3 severe symptoms.

Direct microscopic examination of yeasts and fungal culture were undertaken at day 0 and day 14, at day 7 in case of complete clinical response, and during the follow-up period in the event of relapse. The duration of tablet adhesion and local safety were self-evaluated daily by patients randomized to MBT group using a patient questionnaire. At each visit, any clinical adverse event spontaneously reported by patients and/or observed by investigators or any biological adverse events were reported and graded according to the NCI-CTC scale.

### Endpoints

The primary end-point was the success rate at day 14 defined as complete or partial clinical response where complete clinical response was defined as complete disappearance of oropharyngeal candidiasis lesions and partial clinical response as improvement by at least 2 points of the Murray scoring scale compared with the score at baseline. All other cases were considered as failures. An amendment introduced a blind assessment of the primary criterion carried out in each investigational center by an independent health care member who was unaware of the study drug allocated to each patient. It was implemented after the inclusion of 59 patients.

Secondary endpoints were the success rate at day 7, the percentage of complete response at day 7 and day 14, the percentage of clinical cure (sign and symptom-free patients), the improvement in candidiasis symptoms defined as decrease of at least 1 point in the score coding for the severity of symptoms compared with the score at baseline, the percentage of mycological cure defined as complete eradication of Candida or less than 10 colonies per plate, the relapse rate in responder patients and the progression rate in non responder patients. Time to relapse was calculated. At last, biological, local and general safety was compared between treatments.

Statistical analyses were performed on modified intent-to-treat (mITT; all randomized patients who received at least one treatment dose and had one efficacy evaluation after randomisation) and per protocol (PP) populations. Safety analyses included all patients who received at least one dose of study medication.

A non-inferiority statistical approach was used; a clinical margin of 20% was chosen taking into account that severe local oral conditions could impede the correct adhesion of MBT to the gum. In the absence of data on the efficacy of miconazole oral gel in patients with head and neck cancer who have undergone radiation therapy, a 50% efficacy rate was assumed. A sample size of 103 patients per treatment arm was required to obtain a 90% power in order to demonstrate the non-inferiority of MBT to miconazole gel (α level of 5%, β level of 20%, 2 one-sided). In order to compensate for potential 10% drop outs, and the 59 patients included before the implementation of the abovementioned amendment, the number of patients to be enrolled was increased to 286. Continuous data were compared using either student t-test or Mann Whitney non-parametric test. For binary data, the two treatment groups were compared using Chi square (Chi²) test or Fisher's exact test. The Mantel Haenszel test was used to evaluate ordinal quantitative data.

### Example 5

### Results

After 14 days of treatment, the success rate in the MBT population was statistically not inferior to (p<0.0001) and numerically higher than that in the gel group (56% vs 49%, p<0.0001). Non-inferiority margin was 4.8%. Per protocol and sensitivity analyses confirmed the robustness of the data. Complete responders accounted for almost all the success rate.

As extent of lesions and severely impaired salivary secretions were unevenly distributed, a complementary statistical analysis adjusting for these parameters was performed. It showed a strong trend towards superiority in favour of MBT (p=0.13, table 3). After adjustment, success rate at D14 in patients with multiple lesions was obtained in 58.7% with the MBT versus 37.5% with the gel (57% increase, p=0.013). Likewise, in patients without impaired saliva secretion at inclusion, success rate at D14 was 100% in the MBT group versus 40% in the gel group (150% increase, p=0.034) (Table III).

Clinical success at day 7 was achieved in 14% of the MBT group and in 20% of the gel group (non-inferiority: p=0.0016, Cl₉₅—2.8; 15.2%).

Clinical symptoms improved in 70.3% and 76.5% of the patients treated with MBT and gel respectively (p=NS). Among patients with burning/soreness at inclusion, 63.5% and 56% were symptom-free in the MBT and gel groups respectively (p=NS). They were 57.6% and 50% odynophagia-free patients respectively among those who complained of it at baseline (p=NS). Likewise, erythema and inflammation disappeared in more than 50% of patients in both groups and mucositis in 43.6% in the MBT group (vs, 57.6% in MOG, p=NS). At the end of treatment, 39.1% in both treatment groups were sign and symptom-free (clinical cure) (p=NS).

Fourteen days after the end of treatment, relapse occurred in 19% and 12.5% of patients with complete response in the MBT and gel groups respectively. Forty-five days after the end of treatment, the relapse rate was respectively 21.5% and 17%. On average, the time to relapse was 18.75 and 20.60 days post-treatment for MBT and gel respectively. There was no clinical extension of oral lesion to the oesophagus and no systemic spread in either group. Progression rate in non responder patients at day 60 was low, 4.3% vs 3.9% respectively.

Table III below shows the clinical efficacy results after 14 days' treatment with either miconazole 50 mg Mucoadhesive Buccal Tablet or miconazole 500 mg Oral Gel.

TABLE III

| | Miconazole | | | |
|---|---|---|---|---|
| | 50 mg | Miconazole | | |
| | Mucoadhesive | 500 mg Oral | Difference | 95% CI |
| | buccal tablet | Gel | in % | (P value) |
| mITT CR + | 56% | 48.9% | 7.1% | [−19.0, 4.8] |
| (n = PR | | | | P < 0.0001 |
| 282) CR | 52.5% | 45.4% | 7.1% | [−18.7, 4.5] |
| | | | | P < 0.0001 |

CI = confidence interval;
CR = complete response;
mITT = modified intent-to-treat;
PP = per protocol;
PR = partial response

Table IV below shows the clinical efficacy results after 14 days after adjustment on unbalanced baseline parameters (salivary secretion and extent of lesions).

TABLE IV

| | Miconazole 50 mg | | Miconazole 500 mg | | Difference | |
|---|---|---|---|---|---|---|
| | MAB tablet (%)* | | Oral gel (%)* | | in % | p |
| Lesions | | | | | | |
| Unique | 28/49 | (57.1%) | 33/57 | (57.9%) | −1.4% | 0.94 |
| localised | | | | | | |
| multiple | 44/75 | (58.7%) | 24/64 | (37.5%) | 56.6% | 0.013 |
| localised | | | | | | |
| Spread or | 7/17 | (41.2%) | 11/19 | (57.9%) | −28.8% | 0.32 |
| confluent | | | | | | |
| Saliva | | | | | | |
| Normal | 6/6, | (100%) | 2/5 | (40%) | 150% | 0.034 |
| Partial | 59/105 | (56.2%) | 60/116 | (51.7%) | 8.7% | 0.50 |
| Absent | 14/30 | (46.7%) | 6/19 | (31.6%) | 47.8% | 0.30 |

*p = 0.13 between groups after adjustment on lesion extent and saliva secretion (unbalanced at baseline)

### Example VI

### Overall Safety Data

A total of 130 adverse events (69 in MBT and 61 in gel groups, respectively) was spontaneously reported. There were no differences in safety profile between MBT and gel. The incidence of adverse events (29.3 vs 27.2%, respectively) was similar between the two treatment groups. There were no age or gender effects in either treatment group and no differences in the severity of AE between groups (data not shown). Fewer serious adverse events were reported in the MBT treatment group (0.7% vs 4.7% for gel).

Adverse events occurring with an incidence >1% are listed in Table IV. Nausea and vomiting were more frequently associated with MOG and dysgeusia with miconazole 50 mg MBT. However this latter event was exclusively reported in one investigational centre and likely related to radiation therapy.

Local tolerability assessed only in the miconazole 50 mg MBT using self questionnaire was satisfactory. Overall discomfort (5.3%), burning (7.2%), bad taste (7.2%) and pain (1.8%) were reported in few patients. Patients did not report any adverse events specifically related to the MBT formulation or to the duration of adhesion to the gum.

A significant increase in alanine aminotransferase (ALT) was reported in 3 patients treated with miconazole 500 mg gel. There were no reports of increase in ALT in miconazole 50 mg MBT treated patients.

Table V below shows the overall safety data—Number and prevalence of adverse events occurring during 14 days of treatment with miconazole 50 mg mucoadhesive buccal tablet or miconazole 500 mg oral gel according to age, gender and seriousness.

TABLE V

| | Miconazole | | | | | |
|---|---|---|---|---|---|---|
| | 50 mg | | Miconazole | | | |
| | mucoadhesive | | 500 mg | | | |
| | buccal tablet | | oral gel | | Total | |
| | (n = 147) | | (n = 147) | | (n = 294) | |
| Patients with at least | 43 | (29.2%) | 40 | (27.2%) | 83 | (28.2%) |
| one Adverse Event | | | | | | |
| SAEs | 1 | (0.7%) | 7 | (4.7%) | 8 | (2.7%) |
| Study discontinuation | 3 | (2%) | 6 | (4%) | 9 | (3.06%) |
| due to Adverse | | | | | | |
| Events | | | | | | |
| Deaths | 0 | (0%) | 3 | (2.04%) | 3 | (1.02%) |
| Dysgeusia | 9 | (6.1%) | 0 | | 9 | (3.4%) |
| Abdominal pain | 5 | (3.4%) | 4 | (2%) | 9 | (3.40%) |
| Oral discomfort | 4 | (2.7%) | 5 | (3.4%) | 9 | (3.4%%) |
| Pruritus | 3 | (2%) | 1 | (0.7%) | | |
| Influenza | 3 | (2%) | 3 | (2%) | | |
| Dyspepsia | 2 | | 3 | | 5 | |
| Nausea | 1 | (0.7%) | 4 | (2.7%) | | |
| Vomiting | 1 | (0.7%) | 3 | (2%) | 4 | (1.4%) |
| Infusion site burning | 2 | (1.4%) | 2 | (1.4%) | | |
| Neck pain | 2 | (1.4%) | 1 | (0.7%) | | |
| Bronchitis | 1 | (0.7%) | 3 | (2%) | | |
| Glossodynia | 0 | | 3 | (2%) | 3 | (1%) |
| Toothache | 1 | (0.7%) | 2 | (1.4%) | | |
| Dysphagia | 1 | (0.7%) | 2 | (1.4%) | | |
| Urinary tract infection | 2 | (1.4%) | 0 | | | |
| Asthenia | 2 | (1.4%) | 0 | | | |
| Nasopharyngeal | 1 | (0.7%) | 0 | | | |
| disorder | | | | | | |
| Parotidis | 0 | | 1 | (0.7%) | | |

Despite critical local conditions, tablet adhesion was very good; 92.2% and 61.6% of MBT adhered for 6 and 12 hours, respectively. Approximately 50% of the tablets still adhered to the gum at bedtime. Only 9 (6%) of patients applied less than 11 tablets during the study period and were considered as non compliant. Only 6% tablets were swallowed without occurrence of adverse events. Adhesion for more than 6 hours was slightly less frequent in the first 2 days of treatment (85% of patients) than later (92-100%).

### Discussion

The results of this multicenter, comparative, single blind, randomised study demonstrated that mucosal administration of miconazole 50 mg MBT is an efficacious and safe alternative to systemic oral antifungal agents for the treatment of OPC. Indeed, in this trial, MBT 50 mg administered once daily provided clinical success (complete or partial disappearance of oral lesions) in more than 55% of patients with head and neck cancer patients and a strong trend to superiority over miconazole gel 500 mg administered in 4 divided doses was observed when the statistical analysis was adjusted on baseline salivary secretions and extent of candidiasis lesions that were unbalanced between groups at baseline particularly considering the population with multiple lesions. Clinical cure was observed in about 40% of patients. These results can be compared to those reported with fluconazole administered orally at 50 mg/day for 14 days that induced a clinical response in 53% of patients whereas a clinical cure was obtained in only 21% of patients (23).

In patients with xerostomia, the efficacy of systemic antifungal agents is likely to be low as they are not excreted into saliva and topical treatments are better suited for treating those patients. Our data showed that even if success rates with MBT were lower in these patients (46.7%) than in those with normal (100%) or reduced salivary secretion (56.2%), they were still clinically meaningful and superior to those observed with MOG (36%). This suggests that miconazole is adequately released from MBT and correctly diffused in xerostomic mouth.

There was no relationship between clinical success or clinical cure and mycological cure. This was expected as Candida is a commensal host of oral mucosa and most patients treated for head and neck cancer continue to harbour Candida long after clinical cure. Actually, mycological eradication is not an objective of antifungal treatment, and is rarely achieved even with systemic agents where it is observed in 40-70% depending on the population treated and treatment received (23).

Relapse rates after antifungal treatment have not been reported in patients with head and neck cancer suffering from OPC. Those observed with both MBT and MOG were low and maintained for at least 60 days. Most occurred within 1 month after treatment end. No predictive factors for relapses have been identified. There was no correlation between relapses and the persistence of Candida at the end of treatment. Furthermore, no relationships have been identified between treatment failure or relapse and the occurrence of new Candida species or the emergence of resistant yeasts at the end of the treatment period.

The efficacy of MBT was achieved with a 10-fold lower miconazole dose than that of topical gel (50 mg vs 500 mg) using a once daily administration schedule instead of a four times daily administration. Rapid, high and sustained salivary concentrations are obtained with mucoadhesive tablet (26). Along with a good compliance, they may contribute to a decreased risk of emergence or selection of resistant strains.

Overall, miconazole 50 mg MBT was well tolerated and the nature and severity of the adverse events were in accordance with the pharmacological properties of miconazole. There were no differences in the safety profile between miconazole MBT and gel formulations. Miconazole MBT did not induce increases in liver enzymes that may be deleterious in such patients receiving multiple toxic treatments for their cancer, and at risk of liver metastases.

Miconazole MBT displayed excellent local tolerability with only a small proportion of patients reporting local adverse events and the acceptability of this new treatment was good as demonstrated by the very high compliance rate for a topical treatment. Tablet adhesion to the mucous membrane was excellent, with more than 90% and at least 60% of tablets respectively adhering to the gum for at least 6 and 12 hours. Effective adhesion ensures a continuous release of miconazole and may contribute to the favourable efficacy rate obtained in patients with head and neck cancer and very good patient compliance. Unfortunately, it was not possible to adequately evaluate compliance in the MOG group to verify this assumption.

Lefebvre et al advocated that fluconazole suspension was a useful and well suited therapeutic alternative in patients treated for head and neck cancer because it is administered once daily, well absorbed and easy to swallow (24). MBT shares the same advantages and may be even better suited for these patients as it provided an efficacy rate similar to that reported with systemic oral agents, is well tolerated, may expose these patients at less risk for liver toxicity, drug-drug interactions and may prevent the emergence of resistant strains that have been described with fluconazole (26, 27). MBT provided a better efficacy rate to that reported with local treatment. Then these new results showed a strong trend toward superiority in favour of MBT for local treatment of severe Candidiasis of population with poor prognostic factors response. It may therefore facilitate in clinical practice the compliance with IDSA recommendations to treat OPC with topical agents.

### Example 7

### Clinical Trial Protocol

### Phase III in HIV Positive Patients

A noninferiority, randomized, double-blind, double-dummy, multicenter study comparing miconazole Lauriad® 50 mg bioadhesive buccal tablet once a day for 14 days with clotrimazole troches 10 mg 5 times per day for 14 days was undertaken. The number of patients per treatment group was 270. This number took into account a dropout rate of 10%, with a total of 540 patients enrolled to obtain 270 patients per treatment group. This work was performed at approximately 30 to 35 centers.

HIV positive patients with a clinical picture of oropharyngeal candidiasis characterized by creamy white, curdlike patches on oral mucosal surfaces, which were removable by scraping, or typical erythematous lesions on oral mucosal surfaces were selected for this study. Confirmation of this diagnosis by potassium hydroxide (KOH) tests (direct examination) was undertaken and if the Candida culture was negative despite a positive KOH, the patient was dismissed from this study.

HIV positive patients that were also included in this study were patients with documented human immunodeficiency virus (HIV) seropositivity prior to enrollment. or HIV-positive patients with documentation of viral load and with stable antiretroviral treatment for at least 2 months in the case of treatment initiation or 1 month in the case of treatment modification for reason other than inefficacy, throughout the duration of the antifungal treatment (14 days). In case patients were not on antiretroviral therapy, treatment was not initiated during the antifungal treatment.

Also HIV positive patients were chosen for this study based on the following parameters:
(1) Patients having platelets >100,000/mm³, neutrophils >750/mm³;
(2) Patients with an aspartate transaminase (AST) and an alanine transaminase (ALT) less than or equal to 5 times the normal level (National Cancer Institute Common Toxicity Criteria (NCI/CTC) Grade 0, 1, 2);
(3) Patients that were 18 years of age (or legal age) or older determined to take part in the trial and to observe protocol requirements;
(4) Patients that were in good general state of health defined by a score <2 on the ECOG (Eastern Cooperative Oncology Group) performance status scale as set forth in Table VI below:

TABLE VI
ECOG PERFORMANCE STATUS

| Grade | ECOG |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry out |
| | any work activities. Up and about more than 50% |
| | of waking hours |
| 3 | Capable of only limited selfcare, confined to bed or chair more |
| | than 50% of waking hours |
| 4 | Completely disabled. Cannot carry on any selfcare. Totally |
| | confined to bed or chair |
| 5 | Dead |

(5) If women of child-bearing potential were chosen for this study it was required that they use an effective contraception method (i.e., hormonal contraception or intrauterine device) at least 1 month prior to the start of the study and throughout the entire duration of the study; and
(6) Patients having given written informed consent, dated and signed.

The following patients were excluded from this study:
(1) Pregnant or breast-feeding women;
(2) Patients presenting with a mental deficiency preventing proper understanding of trial protocol requirements;
(3) Prior randomization in this study;
(4) Patients with upper full or partial dentures with an acrylic border in the canine fossa;
(5) Patients with signs or symptoms of systemic candidiasis (candidemia or invasive infection);
(6) Patients with signs or symptoms of esophagitis (i.e., odynophagia, dysphagia) documented by esophageal endoscopy;
(7) Patients with signs and symptoms of perioral lesions (angular cheilitis) only;
(8) Patients who have taken systemic antifungals within the past 15 days;
(9) Patients who have taken local antifungals within the past 7 days;
(10) Patients suffering from milk allergy or with known history of hypersensitivity to one of the components of the products;
(11) Patients with rare hereditary problems of galactose intolerance, lactase enzyme deficiency or glucose galactose malabsorption;
(12) Patients with hepatocellular deficiency defined by INR (PT versus control PT)>1.7;
(13) Patients having concomitant treatment likely to interfere with miconazole such as anti-arrythmics, anticoagulants (anti-vitamin K type, acenocoumarol and warfarin), astemizole, cisapride and phenyloin;
(14) Participation in another therapeutic trial evaluating new drugs or which could interfere with the evaluation of the drug in the study within the preceding 30 days;
(15) Patients not able to complete the diary and not able to adhere with protocol procedures;
(16) Patients undergoing antibiotic treatment at inclusion, except prophylactic antibiotics in the management and care of HIV (such as trimethoprim-sulfamethoxazole ([TMP-SMZ]) and anti-tuberculosis treatments for stable tuberculosis; and
(17) Patients with a life expectancy under 45 days or a clinical condition such that study completion cannot be ensured.

After the patients were chosen as set forth above, miconazole Lauriad® 50 mg bioadhesive buccal tablets were administered to each patient at a dose of one tablet per day for 14 days. The tablet was administered by positioning the tablet with a finger on the upper gum in the slight depression known as the canine fossa at the front top of the gum.

As a comparison clotrimazole troches 10 mg (Mycelex®) were administered to another group of patients only as a lozenge that were slowly dissolved in the patients mouth at a dose of one troche five times per day for 14 days.

The length of treatment for both medications was 14 days.

The patients administered the above drugs were evaluated as follows. Patients were checked for signs and symptoms 17 to 19 days after administration during a “test of cure” (TOC) visit.

Clinical cure was defined as a complete resolution of signs and symptoms at the TOC visit (Days 17-19) with miconazole Lauriad® tablet (50 mg) compared with clotrimazole troches (10 mg) as administered above.

The patients were evaluated based on principal and secondary criterion. The principal criterion was evaluated using a scoring scale (Murray 1997) for the extent of oral lesion as follows:
0=none
1=single, localized
2=multiple, localized
3=extensive or confluent

Also the scoring scale (Murray 1997) for symptoms of oropharyngeal candidiasis (OPC) (soreness/burning) was rated as follows:
0=absent
1=mild
2=moderate
3=severe

The secondary criteria used in this study was based on an efficacy assessment and a safety assessment. The efficacy assessment evaluated the clinical cure at day 7 (days 6-8); the clinical success rate at the TOC visit (days 17-19); the clinical success rate at day 7 (days 6-8); the partial response at the TOC visit (days 17-19); the adhesion time and reasons for discontinued adhesion (obtained from the required patient's diary); the percentage of negative fungal culture alone and combined with full clinical response at the end of treatment; a susceptibility test (microdilution) was performed on pre- and post-treatment saliva samples in nonresponder patients; and the time to relapse.

The safety assessment was based on the rate of and severity of AE(s) (Adverse Events) and SAE(s) (Severe Adverse Events) according to the NCI/CTC scale; the rate of and time to relapse during post treatment follow-up at day 35 (day 35-38); the local tolerability assessed by gum irritation and a site application inspection by an investigator and oral discomfort (an analog visual scale in the required patient's diary); and systemic exposure determined from blood samples collected after 7 doses of miconazole Lauriad® tablet (day 7).

The testing was randomized for both treatment groups.

To evaluate the results, the following statistical methods were utilized. The sample size calculation and hypothesis was based on an expected cure rate of 70% (in the clotrimazole troches group) with a clinically relevant margin of 15%, a sample size of 270 patients for each treatment was required to have 95% power to demonstrate noninferiority of miconazole Lauriade compared with clotrimazole troches (level of 2.5%, 1-sided test). A total of 540 patients (270 per group) were required for recruitment, considering a 10% dropout rate.

The statistical methods that were used in this study were as follows. The co-primary population for efficacy analysis was the per-protocol population (PP) (all randomized patients without major protocol deviations at inclusion or during the study) as well as the intent-to-treat (ITT) population (all randomized patients who took at least one dose of the study medication).

The primary efficacy variable was the clinical cure defined as a complete resolution of signs and symptoms within 3 to 5 days after the end of treatment (at the TOC visit day 17-19). Descriptive statistics was given for the clinical cure at visit 5 (days 17-19) for both treatment groups. A detailed description was also given by countries.

The 1-sided test at the 2.5% significance level was used. A 95% 2-sided confidence interval (CI) of the difference between both groups was calculated. The co-primary analysis was performed on the PP and the ITT populations (missing clinical evaluation was replaced by “clinical failure” in the ITT population). Analysis of the ITT population according to an LOCF (Last Observation Carried Forward) approach was considered as a sensitivity analysis.

For both treatment groups, a detailed description of the secondary efficacy criteria was undertaken. The rate and time to relapse or progression during posttreatment follow-up at day 35 (day 35-38) for both treatment groups was evaluated. Comparison between the 2 groups was performed by X² test (or Fischer's exact test if the conditions of X² was not satisfied).

The ITT population, all randomized patients who took at least one dose of the study medication, was the population used for the safety analysis. Type and incidence of all AEs was tabulated per treatment group.

The results are shown in Table VII Below

TABLE VII

| Treatment Group | |
|---|---|
| Miconazole | |
| Lauriad ® | Clotrimazole |
| MBT 50 mg | Troches 10 mg |

ITT Population

| Baseline Sign: None/Single, | | |
|---|---|---|
| localized | (N = 28) | (N = 31) |
| Clinical Cure | 19 | 30 |
| Proportion Clinically Cured | 0.679 | 0.968 |
| Baseline Sign: Multiple, localized | (N = 164) | (N = 171) |
| Clinical Cure | 102 | 116 |
| Proportion Clinically Cured | 0.622 | 0.678 |
| Baseline Sign: Extensive or | | |
| confluent | (N = 98) | (N = 85) |
| Clinical Cure | 55 | 41 |
| Proportion Clinically Cured | 0.561 | 0.482 |

PP Population

| Baseline Sign: None/Single, | | |
|---|---|---|
| localized | (N = 25) | (N = 28) |
| Clinical Cure | 18 | 27 |
| Proportion Clinically Cured | 0.720 | 0.964 |
| Baseline Sign: Multiple, localized | (N = 136) | (N = 142) |
| Clinical Cure | 97 | 110 |
| Proportion Clinically Cured | 0.713 | 0.775 |
| Baseline Sign: Extensive or | | |
| confluent | (N = 79) | (N = 66) |
| Clinical Cure | 49 | 38 |
| Proportion Clinically Cured | 0.620 | 0.576 |

N = number of patients,
MBT = mucoadhesive buccal tablet,
ITT = intent-to-treat,
PP = per-protocol,
TOC = test of cure

The primary objective of this study was to evaluate the clinical cure of miconazole Lauriad® 50 mg bioadhesive buccal bablets (miconazole Lauriade tablet) compared with clotrimazole troches at the TOC (Test of Cure) visit (days 17-19). By clinical cure means the complete resolution of presenting signs and symptoms (extent score of lesion=0, signs and symptoms=0).

The secondary objective of this study was to evaluate the clinical cure of miconazole Lauriad® tablet compared with clotrimazole troches at day 7 (days 6-8). To evaluate clinical success rate at the TOC visit (days 17-19), defined as clinical cure or clinical improvement. By clinical improvement means that no visible lesion was seen (extent score of lesion=0) and minimal symptoms (soreness/burning <2) was evaluated.

The clinical success rate at day 7 (days 6-8), as well as the partial response at the TOC visit (day 17-19) was evaluated. The partial response was defined as a decrease in the Murray extent of oral lesions score by >1 level and a stable Murray symptoms score. The partial symptom response was defined as a decrease in the Murray symptoms (soreness/burning) score by >1 level and a stable Murray extent of oral lesions score. The partial clinical/symptom response was defined as a decrease in the Murray extent of oral lesions score by >1 level and a decrease in the Murray symptoms (soreness/burning) score by >1 level.

Other objectives of this study were to determine the microbiological cure of miconazole Lauriad® tablet compared with clotrimazole troches at the test of cure (TOC) visit (days 17-19), to determine the rate of relapse at day 35 (days 35-38), to evaluate general and local tolerability and oral discomfort (patient's diary), to assess the duration of adhesion of miconazole Lauriad® 50 mg bioadhesive buccal tablets (miconazole Lauriad® tablet), the number of tablets detached and/or swallowed, the number of tablets replaced, to assess the systemic exposure of miconazole Lauriade tablet by measuring the plasma miconazole concentration at day 7 (days 6-8), to assess the susceptibility of Candida species by microdilution test and to evaluate compliance.

These results indicate that miconazole Lauriad® 50 mg bioadhesive buccal tablets achieved its primary endpoint of noninferiority to clotrimazole tronches in the complete resolution of signs and symptoms of oropharyngeal candidiasis (complete clinical cure) in both ITT and PP populations. All secondary endpoints were met and the products safety was also confirmed.

While the invention has been described in terms of various preferred embodiments, the skilled artisan will appreciate that various modifications, substitutions, omissions and changes may be made without departing from the scope thereof. Accordingly, it is intended that the scope of the present invention be limited by the scope of the following claims, including equivalents thereof.

### REFERENCES

1. Hauman C H, Thompson I O, Theunissen F. Wolfaardt P. Oral carriage of Candida in healthy and HIV-seropositive persons. Oral Surg Oral Med Oral Pathol. 1993; 76(5):570-572.
2. Bodey G P. Candidiasis in cancer patients. Am J. Med. 1984; 77: 13-19.
3. Mooney M A, Thomas I, Sirois D. Oral candidiasis. Int J. Dermatol. 1995; 34(11):759-765.
4. Epstein J B, Polsky B. Oropharyngeal candidiasis: a review of its clinical spectrum and current therapies. Clin Ther 1998; 20(1):40-57.
5. Peterson D E. Oral Candidiasis. Clin Geriatr Med 1992; 8: 513-527.
6. Clarkson J E, Worthington H V, Eden O B. Interventions for treating oral candidiasis for patients with cancer receiving treatment. Cochrane Database Syst Rev. 2004; (1):CD001972.
7. Sonis S T, Elting L S, Keefe D, Peterson D E, Schubert M, Hauer-Jensen M, Bekele B N, Raber-Durlacher J, Donnelly J P, Rubenstein E B; Mucositis Study Section of the Multinational Association for Supportive Care in Cancer; International Society for Oral Oncology. Perspectives on cancer therapy-induced mucosal injury: pathogenesis, measurement, epidemiology, and consequences for patients. Cancer 2004; 100 (9 Suppl):1995-2025.
8. Redding S W, Zellars R C, Kirkpatrick W R, McAtee R K, Caceres M A, Fothergill A W, Lopez-Ribot J L, Bailey C W, Rinaldi M G, Patterson T F. Epidemiology of oropharyngeal Candida colonization and infection in patients receiving radiation for head and neck cancer. J Clin Microbiol 1999; 37(12): 3896-3900.
9. Ramirez-Amador V, Silverman Jr S, Mayer P, Tyler M, Quivey J. Candida colonization and oral candidiasis in patients undergoing oral and pharyngeal radiation therapy. Oral Surg Oral Med Oral Pathol Oral Radiol Endod. 1997; 84: 149-153.
10. Erstein J B, Freilich M M, Le N D. Risk factors for oropharyngeal candidiasis in patients who receive radiation therapy for malignant conditions of the head and neck. Oral Surg Oral Med Oral Pathol. 1993; 76(2):169-74
11. Leung W K, Dassanayake R S, Yau J Y, Jin L J, Yam W C, Samaranayake L P. Oral colonization, phenotypic, and genotypic profiles of candida species in irradiated, dentate, xerostomic nasopharyngeal carcinoma survivors. J Clin Microbiol 2000; 38 (6): 2219-2226.
12. Makkonen T A, Minn H, Jekunen A, Vilja P, Tuominen J, Joensuu H. Granulocyte macrophage-colony stimulating factor (GM-CSF) and sucralfate in prevention of radiation-induced mucositis: a prospective randomized study. Int J Radiat Oncol Biol Phys 2000; 46(3):525-534.
13. Nicolatou-Galitis O, Dardoufas K, Markoulatos P, Sotiropoulou-Lontou A, Kyprianou K, Kolitsi G, Pissakas G, Skarleas C, Kouloulias V, Papanicolaou V, Legakis N J, Velegraki I. Oral pseudomembranous candidiasis, herpes simplex virus-1 infection, and oral mucositis in head and neck cancer patients receiving radiotherapy and granulocyte-macrophage colony-stimulating factor (GM-CSF) mouthwash. J Oral Pathol Med 2001; 30 (8): 471-480.
14. Silverman S, Jr. Oral cancer: complications of therapy. Oral Surg Oral Med Oral Pathol Oral Radiol Endol. 1999; 88(2): 122-126.
15. Grötz K, Genitsariosis S, Vehling D, Al Nawas B. Candida colonization, mucositis and salivary function after head and neck radiotherapy. Support Care Cancer. 2003; 11(11): 717-721.
16. Pappas P G, Rex J H, Sobel J D, Filler S G, Dismukes W E, Walsh T J, Edwards J E. Guidelines for the treatment of candidiasis. Clin Infect Dis. 2004; 38: 161-189.
17. Munoz P, Fernandez-Turegano C P, Alcala L, Rodriguez-Creixems M, Pelaez T, Bouza E. Frequency and clinical significance of bloodstream infections caused by C albicans strains with reduced susceptibility to fluconazole. Diagn Microbiol Infect Dis. 2002; 44(2):163-167.
18. Waltimo T M, Orstavik D, Meurman J H, Samaranayake L P, Haapasalo M P. In vitro susceptibility of Candida albicans isolates from apical and marginal periodontitis to common antifungal agents. Oral Microbiol Immunol 2000; 15(4):245-8.
19. Murray et al Clinical Therapeutics vol. 19 No. 3 (1997).
20. Kuriyama T, Williams D W, Bagg J, Coulter W A, Ready D, Lewis M A. In vitro susceptibility of oral Candida to seven antifungal agents. Oral Microbiol Immunol. 2005; 20(6):349-53.
21. Ellepola A. N., Samaranayake L P. Antimycotic agents in oral candidosis: an overview: 2. Treatment of oral candidosis. Dent Update 27 (4): 165-174 2000.
22. Fothergill A W. Miconazole: a historical perspective. Expert Rev Anti Infect Ther. 2006; 4(2):171-5.
23. Lefebvre J L, Domenge C. A comparative study of the efficacy and safety of fluconazole oral suspension and amphotericin B oral suspension in cancer patients with mucositis. Oral Oncology. 2002; 38: 337-342.
24. Vazquez J A, Skiest D J, Nieto L, Northland R, Sanne I, Gogate J, Greaves W, Isaacs R. A multicenter randomized trial evaluating posaconazole versus fluconazole for the treatment of oropharyngeal candidiasis in subjects with HIV/AIDS. Clin Infect Dis 2006; 42(8):1179-86.
25. Cardot J M, Chaumont C, Dubray C, Costantini D, Aiache J M. Comparison of the pharmacokinetics of miconazole after administration via a bioadhesive slow release tablet and an oral to healthy male and female subjects. Br J Clin Pharmacol. 2004; 58: 345-351.
26. Ruhnke M. Epidemiology of Candida albicans infections and role of non-Candida-albicans yeasts. Curr Drug Targets. 2006; 7:495-504.
27. Bassetti M, Righi E, Tumbarello M, Di Biagio A, Rosso R, Viscoli C. Candida infections in the intensive care unit: epidemiology, risk factors and therapeutic strategies. Expert Rev Anti Infect Ther. 2006; 4: 875-85.
28. Chaumont C, Cardot J M, Dubray C, Costantini D, Aiache J M. Safety, acceptability, and adhesion of a novel bioadhesive slow release tablet of miconazole in a phase I study. J Myc Med. 2003; 13: 13-18.

What is claimed is:

1. A method for local treatment of oropharyngeal candidiasis in immunocompromised patients having severe oral lesions said method comprising administering to a patient in need of such treatment a buccal mucoadhesive tablet having at least a prolonged release and comprising at least one principle active, quantities of natural proteins representing at least 50% by weight of active principle wherein said at least one active principle is miconazole.

2. The method according to claim 1, wherein immunocompromised patients are cancer patients.

3. The method according to claim 1, wherein said prolonged release mucoadhesive tablet have at least one active principle dissolution percentage of more than 70% over 8 hours, and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar.

4. The method according to claim 1, wherein the at least one active principle is in addition to miconazole, a broad spectrum azole in association with a third further active principle selected from a polyene, an analgesic, a salivation agent, an antiseptic, a salivary substitute, an anaesthetic, an anti-inflammatory, an antibiotic, thalidomide or mixture thereof.

5. The method according to claim 4, wherein said broad spectrum azole is selected from the group of clotrimazole, ketoconazole, fluconazole, itraconazole, isoconazole, econazole, saperconazole, geneconazole, terconazole, butoconazole, tioconazole, oxiconazole, bifonazole, fenticonazole, omoconazole, sertaconazole, voriconazole and sulconazole.

6. The method according to claim 3, wherein the alkali metal alkylsulfate is sodium lauryl sulphate or diethylsulphosuccinate.

7. The method according to claim 3, wherein the compression excipients contain corn starch.

8. The method according to claim 1, wherein the at least one active principle is miconazole present in the amount of between 30% to 50% (w %/w %), the natural proteins are milk proteins present in an amount of from 18% to 28% (w %/w %), the alkali metal alkylsulphate is sodium lauryl sulphate present in an amount of 3.5% to 5.50% (w %/w %), the compression excipient is corn starch, the hydrophilic polymer is hydroxypropylmethycellulose and the monohydrate sugar is lactose monohydrate.

9. The method according to claim 1, wherein said prolonged release mucoadhesive tablet has between 10 to 150 mg of miconazole per tablet.

10. The method according to claim 1, wherein said prolonged release mucoadhesive tablet contains 50 mg of miconazole and is administered once daily for fourteen days.

11. The method according to claim 1, wherein said severe oral lesions are multiple or confluent oral lesions.

12. The method according to claim 1, wherein said prolonged release mucoadhesive tablet has salivary concentrations superior to 1 μg/ml for at least 7 hours.

13. The method according to claim 2, wherein said cancer in said patient is cancer of the head and neck.

14. The method according to claim 2, wherein said patient has undergone or is undergoing radiation therapy.

15. The method according to claim 1, wherein said prolonged release mucoadhesive tablet is administered buccaly.

16. A method for a local treatment of relapse of severe oral lesions caused by oropharyngeal candidiasis in a cancer patient having undergone or undergoing radiation therapy, the method comprising administering to those cancer patients in need of such treatment a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole.

17. The method according to claim 16, wherein the at least one active principle is in addition to miconazole, a broad spectrum azole in association with a third further active principle selected from a polyene, an analgesic, a salivation agent, an antiseptic, a salivary substitute, an anaesthetic, an anti-inflammatory, an antibiotic, thalidomide or mixture thereof.

18. The method according to claim 17, wherein said broad spectrum azole is selected from the group of clotrimazole, ketoconazole, fluconazole, itraconazole, isoconazole, econazole, saperconazole, geneconazole, terconazole, butoconazole, tioconazole, oxiconazole, bifonazole, fenticonazole, omoconazole, sertaconazole, voriconazole and sulconazole.

19. The method according to claim 16, wherein the alkali metal alkylsulfate is sodium lauryl sulphate or diethylsulphosuccinate.

20. The method according to claim 16, wherein the compression excipients contain corn starch.

21. The method according to claim 16, wherein the at least one active principle is miconazole present in the amount of between 30% to 50% (w %/w %), the natural proteins are milk proteins present in an amount of from 18% to 28% (w %/w %), the alkali metal alkylsulphate is sodium lauryl sulphate present in an amount of 3.5% to 5.50% (w %/w %), the compression excipient is corn starch, the hydrophilic polymer is hydroxypropylmethycellulose and the monohydrate sugar is lactose monohydrate.

22. The method according to claim 16, wherein said prolonged release mucoadhesive tablet has between 10 to 150 mg of miconazole per tablet.

23. The method according to claim 16, wherein said prolonged release mucoadhesive tablet contains 50 mg of miconazole and is administered once daily for fourteen days.

24. The method according to claim 1, wherein said prolonged release mucoadhesive tablet is administered buccaly.

25. A method of a local treatment of xerostomia in cancer patients having severe oral lesions, said method comprising: administering to those cancer patients in need of such treatment a prolonged release mucoadhesive tablet having at least one active principle dissolution percentage of more than 70% over 8 hours, comprising quantities of natural proteins representing at least 50% by weight of active principle and at least 20% by weight of said prolonged release mucoadhesive tablet, between 10% and 20% of a hydrophilic polymer, compression excipients and comprising between 3.5% and 10% of an alkali metal alkylsulphate and between 0.1% and 1% of a monohydrate sugar, wherein said at least one active principle is miconazole.

26. The method according to claim 25, wherein the at least one active principle is in addition to miconazole, a broad spectrum azole in association with a third further active principle selected from a polyene, an analgesic, a salivation agent, an antiseptic, a salivary substitute, an anaesthetic, an anti-inflammatory, an antibiotic, thalidomide or mixture thereof.

27. The method according to claim 26, wherein said broad spectrum azole is selected from the group of clotrimazole, ketoconazole, fluconazole, itraconazole, isoconazole, econazole, saperconazole, geneconazole, terconazole, butoconazole, tioconazole, oxiconazole, bifonazole, fenticonazole, omoconazole, sertaconazole, voriconazole and sulconazole.

28. The method according to claim 25, wherein the alkali metal alkylsulfate is sodium lauryl sulphate or diethylsulphosuccinate.

29. The method according to claim 25, wherein the compression excipients contain corn starch.

30. The method according to claim 25, wherein the at least one active principle is miconazole present in the amount of between 30% to 50% (w %/w %), the natural proteins are milk proteins present in an amount of from 18% to 28% (w %/w %), the alkali metal alkylsulphate is sodium lauryl sulphate present in an amount of 3.5% to 5.50% (w %/w %), the compression excipient is corn starch, the hydrophilic polymer is hydroxypropylmethycellulose and the monohydrate sugar is lactose monohydrate.

31. The method according to claim 25, wherein said prolonged release mucoadhesive tablet has between 10 to 150 mg of miconazole per tablet.

32. The method according to claim 25, wherein said prolonged release mucoadhesive tablet contains 50 mg of miconazole and is administered once daily for fourteen days.

33. The method according to claim 25, wherein said prolonged release mucoadhesive tablet is administered buccaly.
